(19) United States
(12) Patent Application Publication
(10) Pub. No.: US 2009/0157150 A1
(43) Pub. Date: Jun. 18, 2009

(54) Implanted Driver with Resistive Charge Balancing

(75) Inventor: Lawrence Cauller, Plano, TX (US)

Correspondence Address: GROOVER & Associates, BOX 802889, DALLAS, TX, 75380-2889, US

(73) Assignees: Microtransponder Inc., Dallas, TX (US); The Board of Regents, The University of Texas System, Austin, TX (US)

(21) Appl. No.: 12/323,969

(22) Filed: Nov. 26, 2008

Related U.S. Application Data
(60) Provisional application No. 60/990,278, filed on Nov. 26, 2007.

(51) Int. Cl. A61N 1/08

(52) U.S. Cl. 607/72

(57) ABSTRACT

A transponder includes a stimulus driver configured to discharge an electrical stimulus when a trigger signal is received. A first conducting electrode is coupled to the stimulus driver and conducts the electrical stimulus discharged by the stimulus driver. A second conducting electrode is coupled to the stimulus driver and conducts the electrical stimulus conducted by the first conducting electrode. A depolarization resistance connects the first conducting electrode to the second conducting electrode in response to the trigger signal.

### CROSS-REFERENCE TO ANOTHER APPLICATION

U.S. Provisional Patent Application (Ser. No. 60/990,278 filed 11/26/2007, Attorney Ref MSTP-28P) is hereby incorporated by reference. This application may be related to the present application, or may merely have some drawings and/or disclosure in common.

### BACKGROUND

The present application relates to electrical tissue stimulation devices, and more particularly to a charge-balancing driver circuit.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIG. 1 is a circuit diagram depicting a depolarizing microtransponder driver circuit, in accordance with an embodiment;

FIG. 2 is a graph depicting a stimulus voltage in accordance with an embodiment;

FIG. 3 is a block diagram depicting a microtransponder system, in accordance with an embodiment;

FIG. 4 is a circuit diagram depicting a driver circuit, in accordance with an embodiment;

FIG. 5 is a circuit diagram depicting a driver circuit, in accordance with an embodiment;

FIG. 6 is a circuit diagram depicting a driver circuit, in accordance with an embodiment;

FIG. 7 is a circuit diagram depicting a driver circuit, in accordance with an embodiment; and

FIG. 8 is a circuit diagram depicting a tissue model.

### DETAILED DESCRIPTION OF SAMPLE EMBODIMENTS

Note that the points discussed below may reflect the hindsight gained from the disclosed inventions, and are not necessarily admitted to be prior art.

Human tissue may be stimulated by applying short pulses of electrical energy to the tissue. An electrode pair is positioned proximate to the intended tissue. The electrodes are generally implanted under the skin to provide stimulation to nerve tissue. Typically, a driver circuit connected to the electrodes generates pulses that energize the electrodes. As each pulse generates a voltage drop between the electrodes, current flows along a path through the tissue. The tissue is stimulated when a threshold current flows through the tissue.

Typically, a series of pulses are generated by the driver circuit, at a periodic frequency. When the frequency of these pulses is greater than two cycles per second, the tissue may become polarized. Polarized tissue holds a charge. Because the tissue becomes charged, a larger voltage drop is required to generate the desired stimulation threshold current.

The present application discloses new approaches to a transponder including a stimulus driver configured to discharge an electrical stimulus when a trigger signal is received. A first conducting electrode is coupled to the stimulus driver and conducts the electrical stimulus discharged by the stimulus driver. A second conducting electrode is coupled to the stimulus driver and conducts the electrical stimulus conducted by the first conducting electrode. A depolarization resistance connects the first conducting electrode to the second conducting electrode in response to the trigger signal.

The disclosed innovations, in various embodiments, provide one or more of at least the following advantages. However, not all of these advantages result from every one of the innovations disclosed, and this list of advantages does not limit the various claimed inventions.
charge balancing to depolarize tissue

The numerous innovative teachings of the present application will be described with particular reference to presently preferred embodiments (by way of example, and not of limitation).

A transponder includes a stimulus driver configured to discharge an electrical stimulus when a trigger signal is received. A first conducting electrode is coupled to the stimulus driver and conducts the electrical stimulus discharged by the stimulus driver. A second conducting electrode is coupled to the stimulus driver and conducts the electrical stimulus conducted by the first conducting electrode. A depolarization switch is gated by the trigger signal and connects the first conducting electrode to the second conducting electrode in response to the trigger signal.

Various embodiments describe miniaturized, minimally invasive, wireless implants termed “microtransponders.” Typically, a microtransponder may be sufficiently small that hundreds of independent microtransponders may be implanted under a square inch of skin. These groups or arrays of microtransponders may be used to sense a wide range of biological signals. The microtransponders may be used to stimulate a variety of tissues and may generate a variety of stimulation responses. The microtransponders may be designed to operate without implanted batteries. The microtransponders may be designed so that there is no need for wires to pass through the patient's skin. The microtransponders may be used to treat medical conditions such as chronic pain and similarly.

Microtransponders typically receive energy from the flux of an electromagnetic field. Typically, the electromagnetic field may be generated by pliable coils placed on the surface of the overlying skin. Wireless communication technologies may exploit near-field magnetic coupling between two simple coils tuned to resonate at the same or related frequencies. References to tuning a pair of coils to the “same frequency” may include tuning the pair of coils to harmonically related frequencies. Frequency harmonics make it possible for different, harmonically related, frequencies to transfer power effectively.

By energizing a coil at a related frequency, for example, a selected radio frequency, an oscillating electromagnetic field will be generated in the space around the coil. By placing another coil, tuned to resonate at the same selected radio frequency, in the generated oscillating electromagnetic field, a current will generated in the coil. This current may be detected, stored in a capacitor and used to energize circuits.

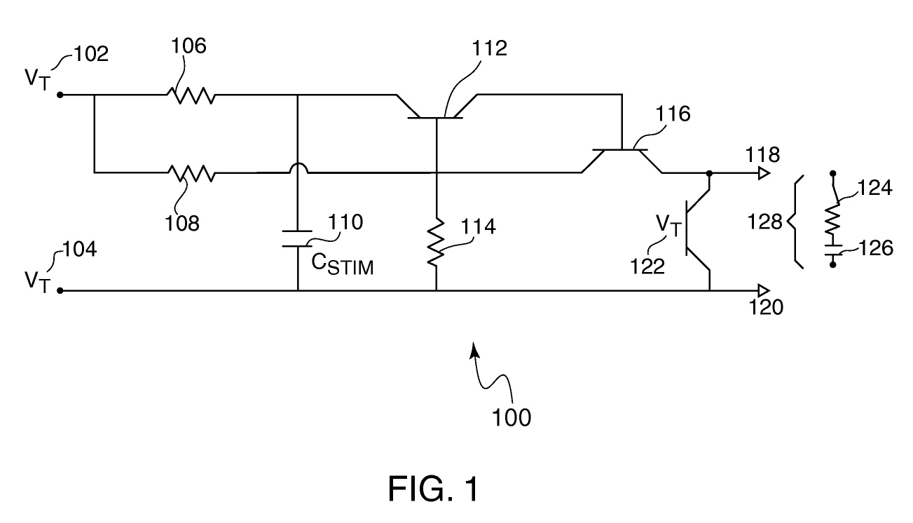

With reference to FIG. 1, a schematic diagram depicts a depolarizing microtransponder driver circuit 100 in accordance with an embodiment. An oscillating trigger voltage (VT and −VT) may be applied between the input nodes 102 and 104 of the driver circuit 100. An auto-triggering microtransponder may employ a bi-stable switch 112 to oscillate between the charging phase that builds up a charge on the stimulus capacitor CSTIM 110 and the discharge phase that can be triggered when the charge reaches the desired voltage and closes the switch 112 to discharge the capacitor 110 through stimulus electrodes 118 and 120.

A resistor 106 regulates the stimulus frequency by limiting the charging rate. The stimulus peak and amplitude are largely determined by the effective tissue resistance 128, modeled with a resistance 124 and a capacitance 126. As such, the stimulus is generally independent of the applied RF power intensity. On the other hand, increasing the RF power may increase the stimulation frequency by reducing the time it takes to charge up to the stimulus voltage.

When a stimulation signal is applied to living tissue at frequencies higher than two hertz, the tissue typically becomes polarized, exhibiting an inherent capacitance 126 by storing a persistent electrical charge. In order to reduce the polarization effect, a depolarization switch 122 is connected between the electrodes 118 and 120. The gate terminal of the depolarization switch 122 is connected to the oscillating trigger voltage VT, so that once each cycle, the depolarization switch shorts the electrodes 118 and 120 and reduces the charge stored in the inherent tissue capacitance 126. The timing of the depolarization switch 122 permits the stimulation pulse to be substantially discharged before the depolarization switch 122 closes and shorts the electrodes 118 and 120. Similarly, the depolarization switch 122 is timed to open before a subsequent stimulation pulse arrives. The timing of the depolarization switch 122 may be generated relative to the timing of the stimulation pulse, The timing may be accomplished using digital delays, analog delays, clocks, logic devices or any other suitable timing mechanism.

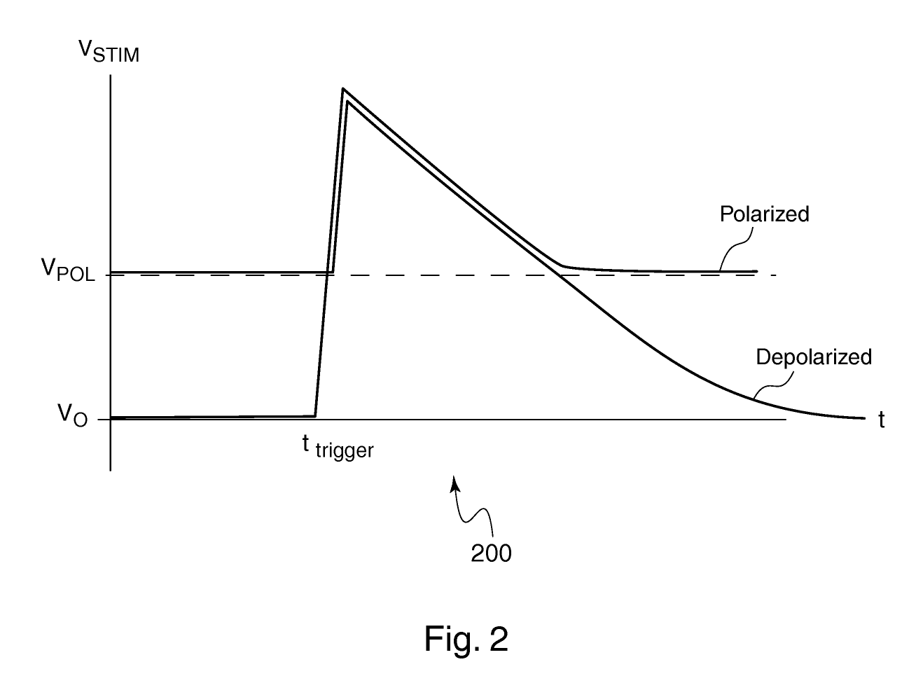

With reference to FIG. 2, a graph depicts an exemplary stimulus discharge in accordance with an embodiment. When a trigger signal is received, the stimulus capacitor discharges current between the electrodes. Depending on the tissue resistance, the voltage quickly returns to a rest voltage level at approximately the initial voltage level. When the frequency of the trigger signal is increased, a polarization effect causes the rest voltage to rise to a polarization voltage above the initial voltage. With a depolarization switch between the electrodes, each trigger signal causes the rest voltage to be re-established and lowered to about the initial voltage level.

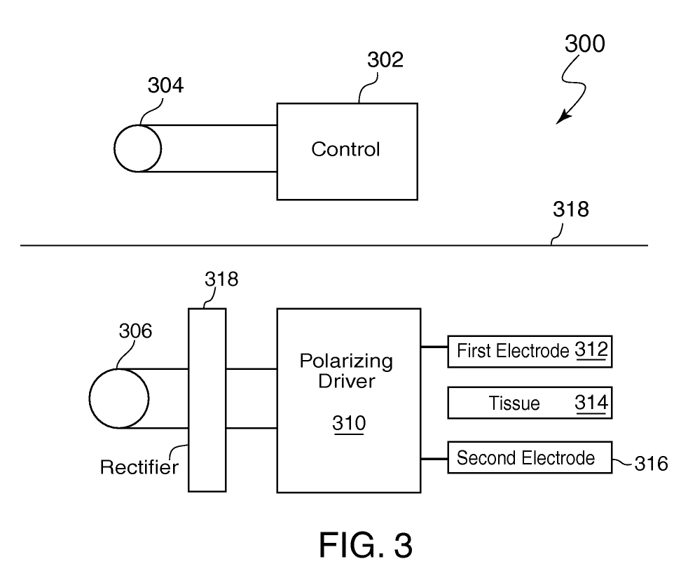

With reference to FIG. 3, a block diagram depicts a depolarizing microtransponder system 300 in accordance with an embodiment. A control component energizes an external resonator element 304 positioned externally relative to an organic layer boundary 318. Energized, the external resonator element 304 resonates energy at a resonant frequency, such as a selected RF. Internal resonator element 306, positioned internally relative to an organic layer boundary 318, is tuned to resonate at the same resonant frequency, or a harmonically related resonant frequency as the external resonator element 304. Energized by the resonating energy, the internal resonator element 306 generates pulses of energy rectified by a rectifier 318. The energy may typically be stored and produced subject to timing controls or other forms of control. The energy is provided to the depolarizing driver 310. A first electrode 312 is polarized relative to a second electrode 316 so that current is drawn through the tissue 314 being stimulated, proximate to the electrode 312 and 316. The first electrode 312 is polarized relative to the second electrode 316 in the opposite polarization to draw an oppositely directed current through the tissue 314, depolarizing the tissue 314. The electrodes 312 and 316 may be typically made of gold or iridium, or any other suitable material.

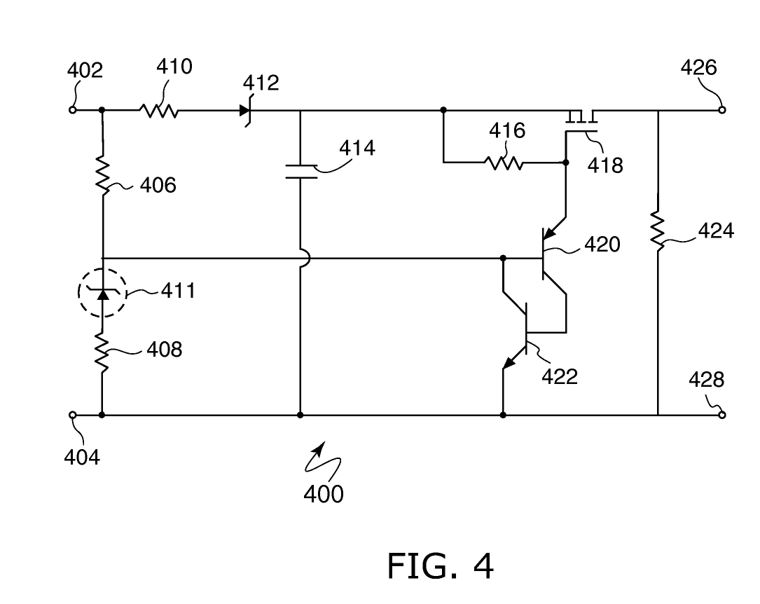

With reference to FIG. 4, a circuit diagram depicts a depolarization driver circuit 400, in accordance with an embodiment. A trigger signal is applied between electrodes 402 and 404. A charge capacitance 414 is charged on the charge capacitance 414. Schottky diode 412 prevents the backflow of stimulus charge during the trigger phase. The charge rate is regulated by resistances 410, 406 and 408. Resistances 406 and 408 form a voltage divider so that a portion of the trigger signal operate the bipolar switches 420 and 422. The trigger signal closes CMOS 418 through resistance 416, connecting the pulse between electrodes 426 and 428. A depolarization resistance 424 is connected between the electrodes 426 and 428 to balance the charge stored in the tissue between the electrodes 426 and 428 between pulses. Because the resistivity of the tissue is non-linear, the time constant of the depolarization resistance must be significantly longer than the time constant of the stimulation pulses. The specific breakdown voltage of the optional Zener diode 411 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 411 sets the maximum stimulus voltage. Otherwise the stimulus voltage is a function of the RF power level reaching the transponder from the external reader coil when the stimulus is triggered.

Differential impedance: in discussing a nonlinear impedance, the linear Ohm's Law relation R=E/I cannot be used. One way to analyze the behavior of some nonlinear impedances is to locally approximate the slope of the E v. I curve, so that differential impedance can be defined as R′(v)=dV/dI at a voltage value v.

The particular importance of this in neurostimulation is that the tissue's impedance is very nonlinear: at full pulse height, e.g. when 10V or so is applied across electrodes which are only separated by a millimeter or so, the differential impedance of tissue is much larger than it is when the pulse voltage has faded to a volt or so. The difference can be an order of magnitude or more.

The present inventor has realized that this relation of the differential impedances of tissue permits a very surprising approach to reducing the residual polarization of tissue: a high-value clamping resistor (e.g. 100 kilohms, in the implementation described is left connected across the output terminals. This resistor is selected to be significantly higher than the differential impedance at full pulso voltage, so that not much of the pulse is dissipated in the resistor. However, the resistor is also preferably comparable to or smaller than the tissue impedance at smaller voltages, so that the resistor provides a DC path to discharge the polarization on the stimulation terminals. This resistor is preferably built into the stimulation circuit, but could alternatively be integrated into the same package.

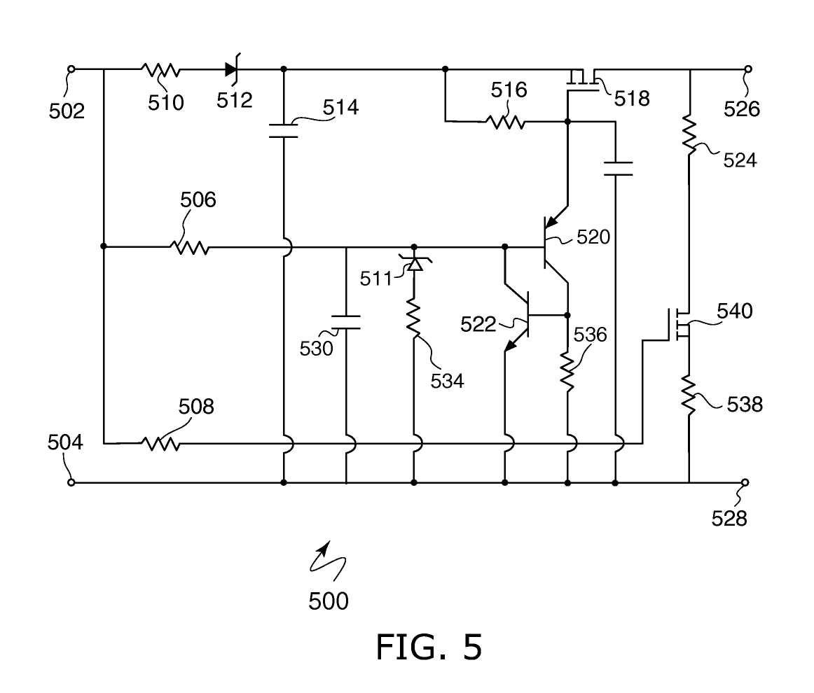

With reference to FIG. 5, a circuit diagram depicts a depolarization driver circuit 500, in accordance with an embodiment. A trigger signal is applied between electrodes 502 and 504. A charge capacitance 514 is charged on the charge capacitance 514. Schottky diode 512 prevents the backflow of stimulus charge during the trigger phase. The charge rate is regulated by resistances 510, 506, 534 and 508. Resistances 506 and 508 form a voltage divider so that a portion of the trigger signal operate the bipolar switches 520 and 522. The trigger signal closes CMOS 518 through resistance 516, connecting the pulse between electrodes 526 and 528. Depolarization resistances 524 and 538 are connected to a depolarization CMOS 540 between the electrodes 526 and 528 to balance the charge stored in the tissue between the electrodes 526 and 528 between pulses. The specific breakdown voltage of the optional Zener diode 511 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 511 sets the maximum stimulus voltage. Otherwise the stimulus voltage is a function of the RF power level reaching the transponder from the external reader coil when the stimulus is triggered.

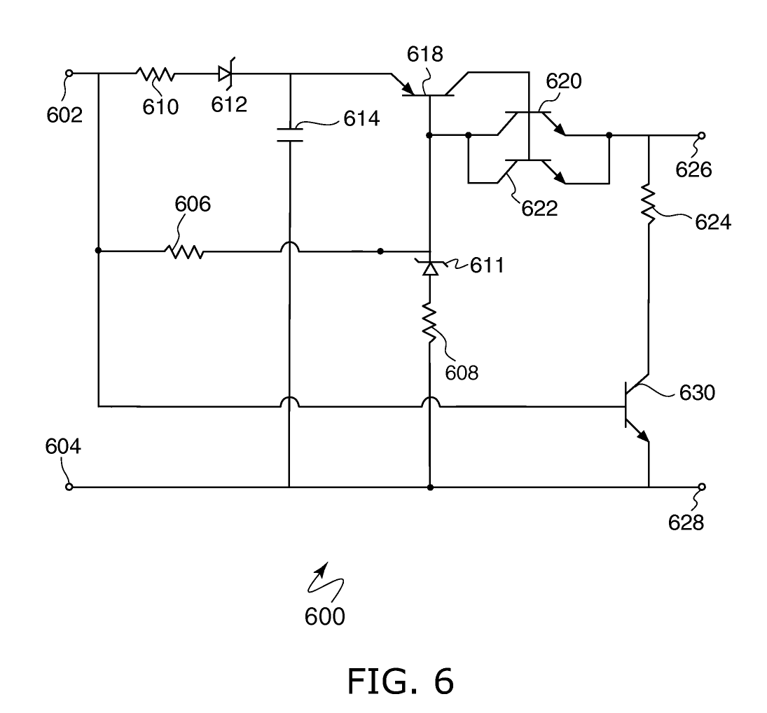

With reference to FIG. 6, a circuit diagram depicts a depolarization driver circuit 600, in accordance with an embodiment. A trigger signal is applied between electrodes 602 and 604. A charge capacitance 614 is charged on the charge capacitance 614. Schottky diode 612 prevents the backflow of stimulus charge during the trigger phase. The charge rate is regulated by resistances 610, 606 and 608. Resistances 606 and 608 form a voltage divider so that a portion of the trigger signal operate the bipolar switches 620 and 622. The trigger signal closes switch 618 through resistance 616, connecting the pulse between electrodes 626 and 628. A depolarization resistance 624 is connected to a bipolar switch 630 between the electrodes 626 and 628 to balance the charge stored in the tissue between the electrodes 626 and 628 between pulses. The specific breakdown voltage of the optional Zener diode 611 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 611 sets the maximum stimulus voltage. Otherwise the stimulus voltage is a function of the RF power level reaching the transponder from the external reader coil when the stimulus is triggered.

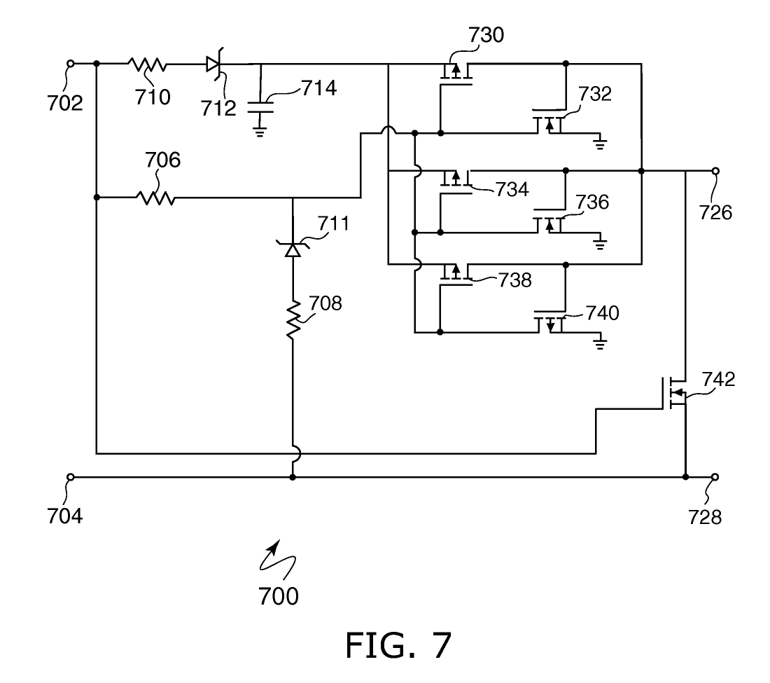

With reference to FIG. 7, a circuit diagram depicts a depolarization driver circuit 700, in accordance with an embodiment. A trigger signal is applied between electrodes 702 and 704. A charge capacitance 714 is charged on the charge capacitance 714. Schottky diode 712 prevents the backflow of stimulus charge during the trigger phase. The charge rate is regulated by resistances 710, 706 and 708. Resistances 706 and 708 form a voltage divider so that a portion of the trigger signal operate the CMOS switches 730, 732, 734, 736, 738 and 740. The trigger signal closes CMOS 730, 734 and 736 connecting the pulse between electrodes 726 and 728. A depolarization CMOS 742 is connected between the electrodes 726 and 728 to balance the charge stored in the tissue between the electrodes 726 and 728 between pulses. The specific breakdown voltage of the optional Zener diode 711 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 711 sets the maximum stimulus voltage. Otherwise the stimulus voltage is a function of the RF power level reaching the transponder from the external reader coil when the stimulus is triggered.

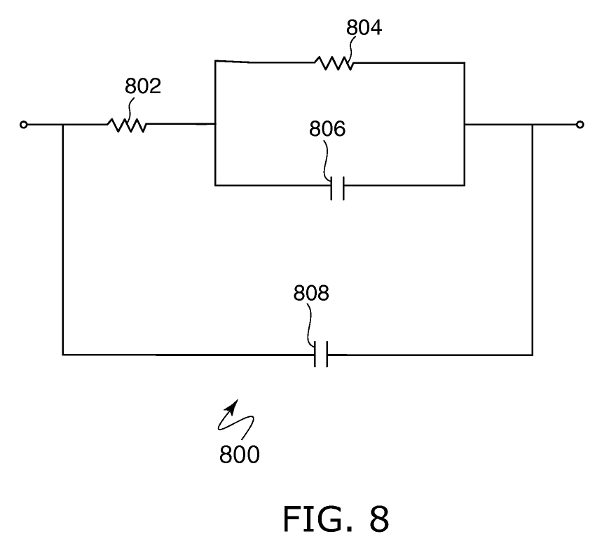

With reference to FIG. 8, a circuit diagram depicts a tissue model. Depolarization becomes important because the tissue behaves as a non-linear load that can be modeled as shown. A resistance 802 is in series with a resistance 804 in parallel with a capacitance 806. This arrangement is parallel to a second capacitance 808. The capacitances and 808 result in charge being stored in the circuit when an intermittent signal is applied, as happens in the tissue being stimulated by intermittent stimulation signals.

### Modifications and Variations

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

A voltage booster may be inserted immediately after the rectifier element 318 to boost the supply voltage available for stimulation and operation of integrated electronics beyond the limits of what might be generated by a miniaturized LC resonant tank circuit. The voltage booster may enable electro-stimulation and other microtransponder operations using the smallest possible LC components, which may generate too little voltage, for example, less than 0.5 volts.

Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application that the microtransponder is applied to.

According to various embodiments, there is provided a method of providing stimulation pulses to tissue comprising providing stimulation pulses to said tissue; and reducing polarization in said tissue.

According to various embodiments, there is provided a wireless stimulation method comprising wirelessly powering an implanted electronic unit; using said implanted unit to provide stimulation pulses to surrounding tissue, over a voltage range in which said tissue has nonlinear impedance; and reducing polarization of said tissue by dampening said pulses with a resistive path, in the implanted electronic unit, which has a real resistance component which is LARGER than the magnitude of differential impedance of the tissue at the full amplitude of said pulses, and SMALLER than the magnitude of differential impedance of the tissue when the amplitude of said pulses is at 10% of its maximum.

According to various embodiments, there is provided a stimulation driver comprising biocompatible electrodes receiving discontinuous stimulation pulses to tissue; and means for depolarizing said tissue.

According to various embodiments, there is provided a stimulation driver to provide discontinuous stimulation pulses to cellular matter comprising biocompatible electrodes receiving discontinuous stimulation pulses; a resistive connection between said biocompatible electrodes and having a time constant such that polarization of the cellular matter is reduced between said discontinuous stimulation pulses.

According to various embodiments, there is provided a transponder includes a stimulus driver configured to discharge an electrical stimulus when a trigger signal is received. A first conducting electrode is coupled to the stimulus driver and conducts the electrical stimulus discharged by the stimulus driver. A second conducting electrode is coupled to the stimulus driver and conducts the electrical stimulus conducted by the first conducting electrode. A depolarization resistance connects the first conducting electrode to the second conducting electrode in response to the trigger signal.

The following applications may contain additional information and alternative modifications: Attorney Docket No. MTSP-29P, Ser. No. 61/088,099 filed Aug. 12, 2008 and entitled “In Vivo Tests of Switched-Capacitor Neural Stimulation for Use in Minimally-Invasive Wireless Implants; Attorney Docket No. MTSP-30P, Ser. No. 61/088,774 filed Aug. 15, 2008 and entitled “Micro-Coils to Remotely Power Minimally Invasive Microtransponders in Deep Subcutaneous Applications”; Attorney Docket No. MTSP-31P, Ser. No. 61/079,905 filed Jul. 8, 2008 and entitled “Microtransponders with Identified Reply for Subcutaneous Applications”; Attorney Docket No. MTSP-33P, Ser. No. 61/089,179 filed Aug. 15, 2008 and entitled “Addressable Micro-Transponders for Subcutaneous Applications”; Attorney Docket No. MTSP-36P Ser. No. 61/079,004 filed Jul. 8, 2008 and entitled “Microtransponder Array with Biocompatible Scaffold”; Attorney Docket No. MTSP-38P Ser. No. 61/083,290 filed Jul. 24, 2008 and entitled “Minimally Invasive Microtransponders for Subcutaneous Applications” Attorney Docket No. MTSP-39P Ser. No. 61/086,116 filed Aug. 4, 2008 and entitled “Tintinnitus Treatment Methods and Apparatus”; Attorney Docket No. MTSP-40P, Ser. No. 61/086,309 filed Aug. 5, 2008 and entitled “Wireless Neurostimulators for Refractory Chronic Pain”; Attorney Docket No. MTSP-41P, Ser. No. 61/086,314 filed Aug. 5, 2008 and entitled “Use of Wireless Microstimulators for Orofacial Pain”; Attorney Docket No. MTSP-42P, Ser. No. 61/090,408 filed Aug. 20, 2008 and entitled “Update: In Vivo Tests of Switched-Capacitor Neural Stimulation for Use in Minimally-Invasive Wireless Implants”; Attorney Docket No. MTSP-43P, Ser. No. 61/091,908 filed Aug. 26, 2008 and entitled “Update: Minimally Invasive Microtransponders for Subcutaneous Applications”; Attorney Docket No. MTSP-44P, Ser. No. 61/094,086 filed Sep. 4, 2008 and entitled “Microtransponder MicroStim System and Method”; Attorney Docket No. 28, Ser, No. ______, filed ______, and entitled “Implantable Transponder Systems and Methods”; Attorney Docket No. MTSP-30, Ser. No. ______, filed ______ and entitled “Transfer Coil Architecture”; Attorney Docket No. MTSP-31, Ser. No. ______, filed ______ and entitled “Implantable Driver with Charge Balancing”; Attorney Docket No. MTSP-32, Ser. No. ______, filed ______ and entitled “A Biodelivery System for Microtransponder Array”; Attorney Docket No. MTSP-47, Ser. No. ______, filed ______ and entitled “Array of Joined Microtransponders for Implantation”; and Attorney Docket No. MTSP-48, Ser. No. ______, filed ______ and entitled “Implantable Transponder Pulse Stimulation Systems and Methods” and all of which are incorporated by reference herein.

The claims as filed are intended to be as comprehensive as possible, and NO subject matter is intentionally relinquished, dedicated, or abandoned.

1. A method of providing stimulation pulses to tissue, comprising the steps of:
providing stimulation pulses to said tissue: and
reducing polarization in said tissue.

2. A wireless stimulation method comprising:
wirelessly powering an implanted electronic unit;
using said implanted unit to provide stimulation pulses to surrounding tissue, over a voltage range in which said tissue has nonlinear impedance; and
reducing polarization of said tissue by dampening said pulses with a resistive path, in the implanted electronic unit, which has a real resistance component which is larger than the magnitude of differential impedance of the tissue at the full amplitude of said pulses, and smaller than the magnitude of differential impedance of the tissue when the amplitude of said pulses is at 10% of its maximum.

3. (canceled)

4. A stimulation driver to provide discontinuous stimulation pulses to cellular matter comprising:
biocompatible electrodes receiving discontinuous stimulation pulses;
a resistive connection between said biocompatible electrodes and having a time constant such that polarization of the cellular matter is reduced between said discontinuous stimulation pulses.

5. The method of claim 1, wherein reducing polarization includes a depolarization switch connected between two electrodes.

6. The method of claim 5, wherein the depolarization switch comprises at least one bipolar switch.

7. The method of claim 5, further comprising the step of:
shorting the electrodes at least once each cycle; and
timing the depolarization switch to permit the stimulation pulses to be substantially discharged before closing the depolarization switch.

8. The method of claim 5, further comprising the step of:
shorting the electrodes at least once each cycle; and
timing the depolarization switch to open before a subsequent stimulation pulse arrives.

9. The method of claim 1, further comprising the step of:
connecting a high-value clamping resistor across a set of output terminals, wherein the resistor impedance is higher than a differential impedance at full pulse power and provides a direct current path to discharge the polarization into the terminals.

10. The method of claim 1, further comprising the step of:
reducing polarization of said tissue by dampening said pulses with a resistive path, in an implanted electronic unit, which has a real resistive component which is larger than the magnitude of differential impedance of the tissue at full amplitude of said pulses, and smaller than the magnitude of differential impedance of the tissue when the amplitude of said pulses is at 10% of its maximum.

11. The method of claim 1, further comprising the steps of:
providing the stimulation pulses to surrounding tissue over a voltage range in which said tissue has nonlinear impedance.

12. The method of claim 2, wherein reducing polarization includes a depolarization switch connected between two electrodes.

13. The method of claim 2, further comprising the step of:
shorting the electrodes at least once each cycle; and
timing the shorting to permit the stimulation pulses to be substantially discharged before closing the resistive path.

14. The method of claim 2, further comprising the step of:
shorting the electrodes at least once each cycle; and
timing the shorting to open the path before a subsequent stimulation pulse arrives.

15. The method of claim 2, further comprising the step of:
connecting a high-value clamping resistor across a set of output terminals, wherein the resistor impedance is higher than a differential impedance at full pulse power and provides a direct current path to discharge the polarization into the terminals.

16. The method of claim 2, wherein the resistive path comprises at least one bipolar switch.

17. The driver of claim 4, wherein the resistive connection comprises a depolarization switch timed to permit the stimulation pulses to be substantially discharged before closing the depolarization switch.

18. The driver of claim 4, wherein the resistive connection comprises a depolarization switch timed to open before a subsequent stimulation pulse arrives.

19. The driver of claim 4, further comprising a high-value clamping resistor connected across a set of output terminals, wherein the resistor impedance is higher than a differential impedance at full pulse power and provides a direct current path to discharge the polarization into the terminals.

20. The driver of claim 4, further comprising the resistive connection dampening said pulses to reduce polarization of said tissue in an implanted electronic unit, which has a real resistive component which is larger than the magnitude of differential impedance of the tissue at full amplitude of said pulses, and smaller than the magnitude of differential impedance of the tissue when the amplitude of said pulses is at 10% of its maximum.
